(19) United States
(12) Patent Application Publication
(10) Pub. No.: US 2006/0153912 A1
(43) Pub. Date: Jul. 13, 2006

(54) Choline ascorbate formulations

(75) Inventors: Andreas Habich, Speyer (DE); Morten M Hansen, Alleroed (DK); Carsten L Hansen, Herlev (DK); Brigitte Yde, Farum (DK); Alfred Oftring, Bad Duerkheim (DE); Michael Schoenherr, Frankenthal (DE); Felicitas Guth, Mannheim (DE); Oliver Hasselwander, Landau (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 10/528,697

(22) PCT Filed: Sep. 22, 2003
(86) PCT No.: PCT/EP03/10535

§ 371 Date: Sep. 29, 2005

(30) Foreign Application Priority Data
Sep. 24, 2002 (DE) 102 44 397.1

(51) Int. Cl. A61K 9/20; A61K 38/05; A61K 31/375; A61K 31/198

(52) U.S. Cl. 424/464; 514/474; 514/562; 514/18

(57) ABSTRACT

The invention relates to novel choline ascorbate-containing formulations; processes for their preparation and their use in human or animal foods, or human or animal food supplements or pharmaceuticals.

The invention relates to novel choline ascorbate-containing formulations; processes for their preparation and their use in human or animal foods or human or animal food supplements or pharmaceuticals.

Choline {[(H₃C)₃N⁺—CH₂—CH₂—OH]OH⁻} is the basic component of phospholipids of the phosphoglyceride type and is found widely in the vegetable and animal kingdoms. Choline acts as an important factor in biochemical processes, e.g. in methylations. Deficiency thereof leads to fatty degeneration of the liver in animals.

Choline is employed principally in the form of choline chloride or choline bitartrate in drug products for arteriosclerosis and liver parenchymal damage. In livestock nutrition, choline chloride is an important animal food additive.

Choline salts of organic acids, such as, for example, the abovementioned choline bitartrate, or choline salicylate, choline hydrogen citrate, and choline ascorbate are described inter alia in EP-A-0 812 821.

Low-odor choline chloride, bitartrate and dihydrogen citrate with a trimethylamine content of less than 0.2 ppb are described in WO-A-00/48986.

WO-A-91/15198 discloses solid choline chloride formulations in which solid choline chloride is provided with a wax coating. The β-polymorphic form of glyceryl tristearate in particular is proposed as shell material.

Choline ascorbate (CAS) is distinguished by combining two active substances which are important for human and lifestock nutrition—choline and L-ascorbic acid (vitamin C)—in one molecule.

The synthesis of choline ascorbate is described in U.S. Pat. No. 2,823,166, CH 490322 and FR 1,242,805. The synthesis of a particularly pure crystalline choline ascorbate is described in DE-A-101 090 73.

A particular problem with choline ascorbate is its limited thermal and oxidative stability, which is manifested in particular after a certain time inter alia by the occurrence of discolorations.

Thus, solid choline ascorbate shows for example after only a few days at 40° C. and in the presence of humidity a brownish color on the surface. Similar unwanted discolorations are observed after some time in choline ascorbate solutions.

On the other hand, other choline salts such as, for example, choline bitartrate, but also L-ascorbic acid and other salts such as, for example, sodium ascorbate have distinctly greater color stability.

Little is known about these choline ascorbate decomposition reactions. It may be assumed that oxidative secondary reactions in the ascorbic acid part of the molecule are speeded up through the presence of the quaternary ammonium (choline) counterion, or amine components liberated by thermal eliminations likewise cause intensely colored secondary products of ascorbic acid.

This color instability of choline ascorbate prohibits for example its use in vitamin formulations.

Choline ascorbate, in particular its crystalline form, it not only sensitive to light and air but also highly hygroscopic. In addition, solid crystalline choline ascorbate has poor flow properties so that classification is very complicated.

### BRIEF DESCRIPTION OF THE INVENTION

It is an object of the present invention to provide choline ascorbate formulations which no longer have at least some of the prior art disadvantages described above.

We have found that this object is achieved by providing solid choline ascorbate formulations which, compared with crude, unformulated choline ascorbate, show less sensitivity of the choline ascorbate to one or more of the external stress factors air, light, moisture, temperature, pH, metal, especially heavy metals, etc.

A “choline ascorbate formulation” encompasses for the purposes of the present invention all formulations which comprise choline ascorbate and/or an ascorbate-containing choline salt mixture of a choline salt with ascorbic acid which is different from choline ascorbate and/or of a salt of ascorbic acid. These ascorbate-containing choline salt mixtures may in principle contain choline salt and ascorbic acid or ascorbic acid salt in any molar ratio such as, for example, 1:3 to 3:1 or 1:2 to 2:1; however, essentially equimolar mixing ratios are preferred.

In a first preferred embodiment, the invention relates to a solid, for example particulate, choline ascorbate-containing formulation comprising choline ascorbate and at least one formulation aid, which formulation has a color stability such that a solution of this formulation has, under standard conditions (i.e. a solution of this formulation in water/methanol (1:1) in a proportion of about 10% by weight based on the total weight of the solution; prepared by stirring the formulation in the solvent at room temperature for 15 minutes and, where appropriate, removing undissolved constituents of the formulation),
i) a Gardner color number (determined as specified in DIN-ISO 4630 or ASTM D 1544-80) of <4.5, preferably <4, in particular 0.05 to 3 or 0.1 to 2, and/or
ii) a Hazen color number (determined as specified in DIN-ISO 6271 or ASTM D 1045-68, ASTM D 26349 or ASTM D 1209-69) of <800, preferably <700, in particular 10 to 400 or 20 to 350 or 25 to 300.

A further embodiment of the invention relates to a solid, for example particulate, choline ascorbate-containing formulation comprising choline ascorbate and at least one formulation aid, which formulation does not deliquesce on storage under standard conditions in moist ambient air. In particular, no partial or complete dissolution of the formulation is visually evident in this case. Nor is any liquid phase which is removable by filtration (separable) to be observed after storage. Standard conditions of storage mean in this case storage of the formulation at room temperature (20-25° C.) in a moist gas atmosphere such as, for example, air, with a relative gas humidity (p of about 76%, which is set up over a saturated aqueous sodium chloride solution, for a period of 72 hours.

The invention relates in particular to solid formulations wherein
a) choline ascorbate is surface-coated with an inert coating composition;
b) choline ascorbate is embedded in an inert matrix; or
c) a porous carrier is loaded with choline ascorbate, and the loaded carrier is surface-coated where appropriate with an inert coating composition.

“Inert” means in this context in particular that essentially no interactions impairing the stability of choline ascorbate to discoloration or decomposition are to be observed.

In a preferred embodiment of the invention, the formulation additionally comprises an effective amount of at least one addition which further reduces the tendency to discoloration of choline ascorbate. This addition which reduces the tendency to discoloration of choline ascorbate may for example be mixed with the choline ascorbate or be in the form of a mixed crystal therewith and/or be present in the surface coating, in the inert matrix or in the porous carrier. The stabilizer is preferably present in a proportion of about 0.05 to 30 mol % based on the molar content of choline ascorbate.

Suitable stabilizers are preferably selected from sulfur-containing, phosphorus-containing or boron-containing compounds; carboxylic acids and carboxylic acid derivatives; vitamins and vitamin precursors and derivatives; natural product mixtures; hydroxy- or alkoxyaromatic compounds; reductones; or mixtures thereof.

The sulfur-containing stabilizer is, in particular, selected from cysteine, cystine, N-acetylcysteine, thioglycolate, glutathione, dihydrolipoic acid, lipoic acid, sodium dithionite, methionine and thiourea; and where appropriate salts of these compounds.

The phosphorus-containing stabilizer is, in particular, selected from phosphorous and hypophosphorous acid, and salts thereof. The boron-containing stabilizer is, in particular, phenylboronic acid and salts thereof. The stabilizing carboxylic acid or its derivative is, in particular, selected from uric, lactic, malic, citric and excess ascorbic acid, and ascorbyl palmitate; examples which should be mentioned of suitable derivatives of carboxylic acids are salts or esters such as, for example, C₁-C₁₈-alkyl or -alkenyl esters. The stabilizing vitamins, vitamin precursors and derivatives are preferably selected from alpha-, beta- and gamma-tocopherol, tocotrienol and more water-soluble vitamin E derivatives, such as, for example, vitamin E succinate or phosphate; carotenoids; isoflavones; flavonoids and other naturally occurring polyphenols such as, for example, quercetin, epigallocatechin, gallates, ellagic acid and ferulic acid. A suitable stabilizing natural product mixture is, for example, a rosemary extract or green tea extract as described, for example, in Martinez-Tome, M. et al., J. Food Prot. 2001, 64 (9):1412-9. Stabilizing hydroxy- or alkoxyaromatic compounds are selected from 6-ethoxy-1,2-dihydro-2,2,4-trimethylquinoline (ethoxyquin), t-butylhydroxytoluene and t-butylhydroxyanisole. Hydroxyacetone may be mentioned as example of a suitable reductone.

The stabilizer may also be a functional derivative, having a stabilizing action, of one of the above compounds.

It is also possible to use according to the invention combinations of two or more of the abovementioned stabilizing additives.

It is furthermore possible according to the invention in the case of possible optical isomerisms to use all stereoisomeric forms such as, for example, the L or the D isomer, but also mixtures of stereoisomers, such as racemic mixtures.

Examples which may be mentioned of suitable functional derivatives of the above compounds are salts. Salts of the above stabilizers are, in particular, alkali metal and alkaline earth metal salts such as, for example, sodium and potassium salts.

Preferred additives from the above list are S-containing species such as, in particular, cysteine, N-acetylcysteine, dihydrolipoic acid, glutathione or thioglycolate; and P-containing species such as hypophosphorous or phosphorous acid; and carboxylic acids such as ascorbic acid or the salts or esters thereof.

Formulations of the invention are preferably also such that their choline ascorbate content is in a range from about 5 to 95% by weight based on the total weight of the formulation.

In a further preferred embodiment, coated formulations of the invention are provided with a coating composition which comprises at least one compound selected from:
a) polyalkylene glycols, in particularly polyethylene glycols, for example having a number average molecular weight of about 400 to 15 000, such as, for example, 400 to 10 000;
b) polyalkylene oxide polymers or copolymers, for example having a number average molecular weight of about 4000 to 20 000, in particular block copolymers of polyoxyethylene and polyoxypropylene;
c) substituted polystyrenes, maleic acid derivatives and styrene/maleic acid copolymers;
d) vinyl polymers, in particular polyvinylpyrrolidones, for example having a number average molecular weight of about 7000 to 1 000 000; either alone or in combination with other compounds such as cellulose ethers or starches;
e) vinylpyrrolidone/vinyl acetate copolymers, for example having a number average molecular weight of about 30 000 to 100 000;
f) polyvinyl alcohols, for example having a number average molecular weight of about 10 000 to 200 000, and polyphthalic acid vinyl esters;
g) hydroxypropylmethylcelluloses, for example having a number average molecular weight of about 6000 to 80 000;
h) alkyl (meth)acrylate polymers and copolymers, for example having a number average molecular weight of about 100 000 to 1 000 000, in particular ethyl acrylate/methyl methacrylate copolymers and methacrylate/ethyl acrylate copolymers;
i) polyvinyl acetates, for example having a number average molecular weight of about 250 000 to 700 000, where appropriate stabilized with polyvinylpyrrolidone;
j) polyalkylenes, in particular polyethylenes;
k) aromatic polymers, for example lignins;
l) polyacrylic acids;
m) polyacrylamides;
n) polycyanoacrylates;
o) phenoxyacetic acid/formaldehyde resins;
p) cellulose derivatives such as ethylcellulose, ethylmethylcellulose, methylcellulose, hydroxypropylcellulose, hydroxypropylmethylcellulose, carboxymethylcellulose, cellulose acetate phthalate;
q) animal, vegetable or synthetic fats and modified fats such as, for example, polyglycols, fatty alcohols, ethoxylated fatty alcohols, higher fatty acids; mono-, di- and triglycerices of higher fatty acids, e.g. glycerol monostearate, alkyl aryl ethoxylates and cocomonoethanolamides;
r) animal and vegetable waxes or chemically modified animal and vegetable waxes, such as beeswax, candelilla wax, carnauba wax, montan ester wax and rice germ oil wax, spermaceti, lanolin, jojoba wax, Sasol wax;
s) animal and vegetable proteins such as, for example, gelatin, gelatin derivatives, gelatin substitutes, casein, whey, kerain, soybean protein; zein and wheat protein;
t) mono- and disaccharides, oligosaccharides, polysaccharides such as, for example, hyaluronic acid, pullulan, elsinan, starches, modified starches, and pectins, alginates, chitosan, carrageenan;
u) vegetable oils, such as, for example, sunflower, safflower, cottonseed, soybean, corn germ, olive, rapeseed, linseed, coconut, oil palm kernel oils; synthetic or semisynthetic oils such as, for example, medium chain-length triglycerides or mineral oils; animal oils such as, for example, herring, sardine and whale oils;
v) hardened (hydrogenated or partially hydrogenated) oils/fats such as, for example, of those mentioned above in particular hydrogenated palm oil, hydrogenated cottonseed oil, hydrogenated soybean oil;
w) lacquer coatings such as, for example terpenes, in particular shellac, tolu balsam, Peru balsam, sandarac, and silicone resins;
x) fatty acids, both saturated and mono- and polyunsaturated C₆ to C₂₄ carboxylic acids;
y) silicas
   and mixtures thereof.

The addition of plasticizers or emulsifiers to fats or waxes before the coating may be advantageous where appropriate for improving the flexibility of the film.

In a further preferred embodiment there is provision of formulations in which the choline ascorbate is embedded in a matrix which comprises at least one compound which complies with the above general definition and which is suitable for forming a matrix which is solid for example in the range from about 20 to 100° C. or 30 to 100° C.

A further preferred embodiment relates to choline ascorbate-containing formulations where the choline ascorbate is carried by a preferably porous carrier.

Further preferred embodiments relate to solid choline ascorbate formulations which comprise combinations of the features described above. Thus, for example, formulations based on porous carriers, or formulations which comprise choline ascorbate embedded in a matrix, can additionally be provided with a coating composition in order for example to stabilize choline ascorbate further or in order to confer modified processing properties on the product.

The solid choline ascorbate formulations of the invention can be prepared in diverse ways. Nonlimiting examples of suitable modes of preparation comprise:

a process wherein solid choline ascorbate particles are coated by the latter being
a) sprayed in a fluidized bed with a melt, a solution or a dispersion of a coating composition as defined above, or subjected to a powder coating with the coating composition in a fluidized bed; or
b) coated in a mixer with a melt, a solution or a dispersion of the coating composition, or subjected to a powder coating with the coating composition,

and the coated material obtained in each case where appropriate being dried, cooled and/or freed of coarse fractions;

a process wherein solid choline ascorbate particles are suspended in a melt comprising a (fusible) coating composition as defined above, and the suspension obtained in this way is dispersed and subsequently solidified;

a process wherein solid choline ascorbate particles are dispersed in a lipophilic environment, the solid/oil droplets obtained in this way are emulsified in an aqueous phase, and the emulsion is spray-formulated;

a process wherein choline ascorbate particles are coated by coacervation;

a process wherein an aqueous protective colloid solution is prepared, choline ascorbate is dissolved or dispersed therein, and the resulting mixture is subsequently spray-dried;

a process wherein a choline ascorbate-containing aqueous, aqueous/organic or organic solution is spray-dried in a fluidized bed and, where appropriate, granulated or agglomerated by addition of suitable additives;

a process wherein a solution, emulsion or suspension of choline ascorbate is mixed with a porous carrier and dried where appropriate; or a melt comprising choline ascorbate is applied to the porous carrier;

a process wherein wet granules comprising a choline ascorbate-containing solution or a choline ascorbate-containing melt and a carrier; or wet granules comprising crystalline choline ascorbate are prepared, the wet granules are extruded, where appropriate after-treated, dried and subsequently coated where appropriate;

a process wherein an aqueous solution of choline ascorbate is prepared, the latter is emulsified in a hydrophobic melt, and the emulsion is solidified; and

a process wherein a melt comprising choline ascorbate, where appropriate dispersed in a coating composition and/or where appropriate in the presence of a dusting agent, is atomized in a stream of cold gas; and

a process wherein an aqueous, aqueous-organic or organic solution of choline ascorbate is evaporated to a solid in vacuo, where appropriate in the presence of a carrier and/or of additives. The solid can then, where appropriate with addition of a binder, be agglomerated, granulated, compacted and, where necessary, further reduced in size, classified and, where appropriate, coated with a protective layer.

The above preparation processes can also be employed in particular for preparing formulations which comprise at least one of the abovementioned stabilizers.

Thus, for example, a stabilized choline ascorbate-containing formulation can be prepared by
i) solid choline ascorbate or a mixture of a solid choline salt with solid ascorbic acid and/or solid ascorbic acid salt being mixed with an effective amount of a stabilizing addition as defined above in solid or liquid form; and the mixture being dried where appropriate; or
ii) an effective amount of a stabilizing addition as defined above being dissolved or dispersed in an aqueous, aqueous/alcoholic or alcoholic solution of choline ascorbate or of a mixture of a choline salt with ascorbic acid and/or a salt thereof; and the solution or dispersion being concentrated where appropriate to dryness (preferably to an amorphous solid) or the stabilized formulation being crystallized out of the solution; or
iii) a melt or supercooled melt of choline ascorbate or of a mixture of a choline salt with ascorbic acid and/or an ascorbic acid salt being mixed with an effective amount of at least one stabilizing addition as defined above, and the mixture being solidified where appropriate.

These stabilized solids can then be processed further by use of one of the processes described above.

The invention additionally relates to human or animal foods which, besides conventional ingredients of human or animal foods, comprise a choline ascorbate-containing formulation as defined above in a proportion of about 0.001 to 50% by weight, such as, for example, 0.5 to 40% by weight or 1 to 20% by weight. Human foods according to the invention also include in particular infant food.

The invention further relates to human or animal food supplements which, besides conventional ingredients of human or animal food supplements, comprise a choline ascorbate-containing formulation as defined above in a proportion of about 0.01 to 99.9% by weight, such as, for example, 0.5 to 80% by weight or 5 to 50% by weight.

The invention further relates to pharmaceuticals in solid, liquid or pasty form, which comprise in a pharmaceutically suitable carrier an effective amount, such as, for example, 0.1 to 99.9% by weight, such as, for example 1 to 80% by weight or 5 to 60% by weight, of a choline ascorbate-containing formulation as defined above.

Finally, the invention further relates to the use of a choline ascorbate-containing formulation as defined above for preparing human and animal foods, and human and animal food supplements, or pharmaceuticals.

### DETAILED DESCRIPTION OF THE INVENTION

A) Choline Ascorbate

Choline ascorbate is used in solid, dissolved or molten form in the processes of the invention. Solid choline ascorbate may moreover be in amorphous or crystalline form. A preferred crystalline choline ascorbate is described, for example, in the earlier DE-A-101 090 73.

The crystals described therein show as the most intense line in the 2 Θ-X-ray powder diffractogram in the range between 3.40 and 4.70 Å a line at d=3.80 Å. The crystalline choline ascorbate additionally shows an intensity ratio of the diffraction lines at d=3.80 Å and d=4.55 Å of at least 0.5, preferably at least 0.6, particularly preferably of at least 0.7, and at d=3.80 Å and d=4.67 Å of at least 0.4, preferably at least 0.5, particularly preferably of at least 0.6. Besides the diffraction lines at d=3.80, 4.55 and 4.67 Å, the crystals show further lines at d=3.46, 3.78, 6.91, 8.49 and 10.29 Å.

The choline ascorbate crystals have a purity of >98%, preferably >99%, particularly preferably >99.5%.

This crystalline choline ascorbate is prepared by reacting ascorbic acid with trimethylamine and ethylene oxide, the reaction being carried out in the temperature range from −20° C. to 80° C., preferably −10° C. to 40° C., particularly preferably in the temperature range from 0° C. to 30° C.

The process further comprises carrying out the reaction in water, in a water-miscible organic solvent or in a mixture of water and a water-miscible organic solvent. The water content in the solvent can be between 0 and 50% by weight, preferably between 0 and 10% by weight.

Suitable water-miscible solvents are in particular water-miscible, thermally stable, volatile solvents containing only carbon, hydrogen and oxygen, such as alcohols, ethers, esters, ketones and acetals. The solvents preferably used are those which are at least 10% water-miscible, have a boiling point below 200° C. and/or have fewer than 10 carbons. Methanol, ethanol, n-propanol, isopropanol, 1,2-butanediol 1-methyl ether, 1,2-propanediol 1-n-propyl ether, tetrahydrofuran or acetone are particularly preferably used. Methanol and ethanol may be mentioned as very particularly preferred.

The molar ratio of the reactants trimethylamine:ascorbic acid:ethylene oxide is in the range 0.9-1.1:0.9-1.1:0.9-2.0, preferably in the region of 1:1:1.2, particularly preferably in the region of 1:1:1.05.

The crystallization of choline ascorbate preferably takes place in one of the abovementioned solvents used for the reaction.

It is also possible first to react trimethylamine and ethylene oxide in a water-miscible organic solvent or in a mixture of water and a water-miscible organic solvent at temperatures in the range from −20° C. to 80° C., preferably −10° C. to 40° C., particularly preferably in the temperature range from 0° C. to 30° C., and subsequently to convert this solution into choline ascorbate by adding a stoichiometric amount of ascorbic acid, and then to crystallize out.

As a further possible preparation variant, choline chloride may also be reacted with sodium ascorbate in water, in a water-miscible organic solvent or in a mixture of water and a water-miscible organic solvent at temperatures in the range from −20° C. to 80° C., preferably −10° C. to 40° C., particularly preferably in the temperature range from 0° C. to 30° C., to give crystalline choline ascorbate. The sodium chloride which is formed in this case is filtered off for example before crystallizing out the required product.

As already stated above, the invention also extends to the use of mixtures of choline salts (different from choline ascorbate) with ascorbic acid and/or ascorbic acid salts. Examples of choline salts suitable according to the invention include: choline chloride, choline bitartrate, tricholine citrate, bischoline tartrate, bischoline hydrogen phosphate, choline hydrogen phosphate, bischoline hydrogen citrate, choline dihydrogen citrate, choline gluconate, choline salicylate, choline nicotinate, choline folate and choline carboxymethylcellulose.

Examples of suitable ascorbic acid salts are alkali metal and alkaline earth metal salts, such as sodium ascorbate.

B) Stabilizing Additions

The suitability of a compound as choline ascorbate-stabilizing addition, i.e. one suppressing the tendency to discoloration of choline ascorbate, in formulations of the invention can be determined in a simple manner by testing a choline ascorbate solution for its tendency to discoloration under standardized conditions in the presence of the addition.

The addition preferably comprises at least one stabilizer which has the effect that a 50% by weight aqueous/methanolic solution of choline ascorbate or an ascorbate-containing choline salt mixture as defined above in the presence of a particular amount of the stabilizer such as, for example, 1% by weight, based on the total weight of the solution, under standard conditions (heating at a temperature of 65° C. for a period of 7 hours) has
iii) a Gardner color number (determined as specified in DIN-ISO 4630 or ASTM D 1544-80) of <6.3, preferably <5, in particular 0.05 to 3 or 0.1 to 2 and/or
iv) a Hazen color number (determined as specified in DIN-ISO 6271 or ASTM D 1045-68, ASTM D 263-49 or ASTM D 1209-69) of <1000, preferably <980, in particular 10 to 400 or 20 to 350 or 25 to 300.

In a further preferred embodiment, the addition comprises at least one stabilizer which has the effect that a 10% by weight aqueous/methanolic (preferably 1:1 v/v) solution of choline ascorbate, or a mixture of at least one choline salt with ascorbic acid which is different from choline ascorbate and/or an ascorbate acid salt, in the presence of a particular amount of the stabilizer such as, for example, 1% by weight based on the total weight of the solution, under standard conditions (heating at a temperature of 65° C. for a period of 7 hours), has
i) a Gardner color number (determined as specified in DIN-ISO 4630 or ASTM D 1544-80) of <2.0, preferably <1.5, in particular 0.05 to 1.5 or 0.1 to 1.0, and/or
ii) a Hazen color number (determined as specified in DIN-ISO 6271 or ASTM D 1045-68, ASTM D 263-49 or ASTM D 1209-69) of <300, preferably <250, in particular 10 to 150 or 20 to 100 or 25 to 50.

Stabilizers also suitable according to the invention are those which have a more negative redox potential than ascorbic acid.

Stabilizers which can be used according to the invention are present in the formulations in a proportion of about 0.05 to 30 mol %, preferably about 0.1 to 15 mol % or 0.5 to 10 mol %, in each case based on the molar content of choline ascorbate, or of a choline salt different therefrom (on use of an ascorbate-containing choline salt mixture).

C) Coating Materials

Examples which should be mentioned of suitable polyalkylene glycols a) are: polpropylene glycols and, in particular, polyethylene glycols of varying molecular mass such as, for example, PEG 4000 or PEG 6000, obtainable from BASF AG under the proprietary names Lutrol E 4000 and Lutrol E 6000.

Examples which should be mentioned of above polymers b) are: polyethylene oxides and polypropylene oxides, ethylene oxide/propylene oxide copolymers, and block copolymers composed of polyethylene oxide and polypropylene oxide blocks, such as, for example, polymers which are obtainable from BASF AG under the proprietary name Lutrol F68 and Lutrol F127.

It is possible and advantageous to employ highly concentrated solutions of the polymers a) and b), of up to about 50% by weight, such as, for example, about 30 to 50% by weight, based on the total weight of the solution.

Examples which should be mentioned of above polymers d) are: polyvinylpyrrolidones like those marketed for example by BASF AG under the proprietary name Kollidon or Luviskol. It is possible and advantageous to employ highly concentrated solutions of these polymers having a solids content of about 30 to 40% by weight, based on the total weight of the solution.

An example which should be mentioned of abovementioned polymers e) is: a vinylpyrrolidone/vinyl acetate copolymer which is marketed by BASF AG under the proprietary name Kollidon VA64 or Kollicoat SR. It is possible and particularly advantageous to use highly concentrated solutions of these copolymers, of about 30 to 40% by weight based on the total weight of the solution.

Examples which should be mentioned of above polymers f) are: products like those marketed for example by Hoechst under the proprietary name Mowiol. It is possible and advantageous to employ solutions of these polymers having a solids content in the range from about 8 to 20% by weight.

Examples which should be mentioned of suitable polymers g) are: hydroxypropylmethylcelluloses like those marketed for example by Shin Etsu under the proprietary name Pharmacoat.

Examples which should be mentioned of abovementioned polymers h) are: alkyl (meth)acrylate polymers and copolymers whose alkyl group has 1 to 4 carbon atoms. Specific examples which should be mentioned of suitable copolymers are: ethyl acrylate/methyl methacrylate copolymers which are marketed for example under the proprietary name Kollicoat EMM 30D by BASF AG or under the proprietary name Eudragit NE 30 D by Röhm; and methacrylate/ethyl acrylate copolymers like those marketed for example under the proprietary name Kollicoat MAE 30DP by BASF AG or under the proprietary name Eudragit 30/55 by Röhm. Copolymers of these types can for example be processed according to the invention as 10 to 40% by weight dispersions.

Examples which should be mentioned of above polymers i) are: polyvinyl acetate dispersions which are stabilized with polyvinylpyrrolidone and are marketed for example under the proprietary name Kollicoat SR 30D by BASF AG (solids content of the dispersion about 20 to 30% by weight).

Examples which should be mentioned of suitable cellulose derivatives p) are, in particular, cellulose ethers such as methyl- and ethylcellulose, hydroxypropyl- and hydroxypropylmethylcellulose (HPMC), such as, for example, the commercial products of the Methocel, Bebecel and Pharmacoat series; but also microcrystalline cellulose (MCC) such as, for example, Avicel PH101 or PH 102.

Examples which should be mentioned of suitable saccharides t) are alginates, carrageenan, starch and starch derivatives such as, for example, products of the esterification of starch; gum, such as gum acacia, xanthan gum and guar gum, and gum from Ceratonia siliqua (locust bean gum), hyaluronic acid, pullulan, elsinan.

Examples which should be mentioned of suitable waxes r) are animal waxes such as lanolin, beeswax, and spermaceti; vegetable waxes such as candelilla wax, carnauba wax and rice germ oil wax; and chemically modified waxes such as jojoba wax, Sasol wax and montan ester wax.

Examples of suitable oils u) are vegetable oils such as sunflower, safflower, cottonseed, soybean, corn germ, olive, rapeseed, linseed, coconut, oil palm kernel and oil palm oil; animal oils such as, for example, herring, sardine and whale oil; and hydrogenated products derived therefrom; and semisynthetic oils such as medium chain-length triglycerides and mineral oils. Examples of suitable commercial products are Coatex 01 and 21, Akofine R and Akocote RT

Also suitable are ready-to-use coating compositions such as, for example, Sepifilm LP, consisting of HPMC (70-90%), MCC (8-12%), stearic acid (5-15%) and titanium dioxide (10-20%); or Lustre Clear LC 104, consisting of MCC, carrageenan, lactose, soybean lecithin and propylene glycol alginate.

Protective colloids should be mentioned as an independent group of suitable materials. Synthetic and biological polymers are suitable for this purpose. Examples of synthetic polymers are neutral polymers such as Kollidon, Luviskol, Lutrol and Mowiol, anionic polymers such as Kollicoat, Eudragit L and polyaspartic acid, and cationic polymers such as terpolymer and Eudragit E. Suitable proteinaceous biopolymers are gelatin, casein and whey, soybean protein and wheat protein; suitable polysaccharides are anionic compounds such as gum arabic, HPMC, pectins, alginates, modified starch and shellac; and cationic polysaccharides such as chitosan.

Further suitable coating compositions and coating processes can be found in R. Voigt, Lehrbuch der pharmazeutischen Technologie, 1975, Verlag Chemie, in particular chapters 9.4, 9.5, 9.6 and 10.2.

D) Carriers

The formulations of the invention may also comprise carriers. Conventional inert carriers can be used for example for this purpose. An “inert” carrier must not show any adverse interactions with the components employed in the formulation of the invention and must be acceptable for use as auxiliary in the respective uses, e.g. in human foods, human food supplements, animal foods, animal food additives, pharmaceutical and cosmetic preparations.

Examples which may be mentioned of suitable carrier materials are: low molecular weight inorganic or organic compounds, and high molecular weight organic compounds of natural or synthetic origin.

Examples of suitable low molecular weight inorganic carriers are salts such as sodium chloride, calcium carbonate, sodium sulfate and magnesium sulfate or kieselguhr or silicas such as silicon dioxides or silica gels or silica derivatives such as, for example, silicates.

Examples of suitable organic carriers are, in particular, sugars such as, for example, glucose, fructose, sucrose, dextrins, starch products, especially corn starch and cellulose products. Examples which should be mentioned of other organic carriers are: corncob meal, ground rice husks, wheat bran or cereals flours such as, for example, wheat, rye, barley and oatmeal or bran or mixtures thereof. Further suitable porous carriers are disclosed for example in U.S. Pat. No. 6,251,478, as are processes for loading such carriers. The disclosure of this publication is incorporated herein by reference.

The carrier material may be present in the formulation of the invention in a proportion of about 5 to 95% by weight, preferably about 10 to 85% by weight, on a dry basis.

The particle size of the carrier can be for example in the range from about 30 to 2500 μm, such as, for example, 50 to 2000 μm.

Adsorbates of the invention are preferably based on silica carriers.

E) Further Additives

Besides the ingredients described above, such as choline ascorbate, carrier, stabilizer and coating composition, the formulations of the invention may comprise further additions. Examples which may be mentioned are preservatives, antibiotics, antimicrobial additions, antioxidants, chelating agents, physiologically acceptable salts, flavorings, colors and the like. Additions relevant to nutrition may also be present, such as, for example, vitamins (e.g. vitamins A, B₁, B₂, B₆, B₁₂, C, D₃ and/or E, K₃, folic acid, nicotinic acid, pantothenic acid); taurine, carboxylic acids and salts thereof, such as, for example, tricarboxylic acids such as citrate, isocitrate, trans/cis-aconitate and/or homo-citrate, enzymes, carotenoids, minerals, such as, for example, P, Ca, Mg and/or Fe, and trace elements such as Se, Cr, Zn, Mn, proteins, carbohydrates, fats, amino acids. It is also possible for pyruvic acid, L-carnitine, lipoic acid, coenzyme Q10, amino carboxylic acids such as, for example, creatine, orotic acid, myoinositol, flavonoids, betaine, p-aminobenzoic acid to be present.

It is additionally possible for “active ingredients” which assist the use of the formulation of the invention in pharmaceutical preparations or whose effect serves to treat diseases, especially to treat cancer, diabetes, AIDS, allergies and cardiovascular disorders, to be present.

The above additions, including carriers, coating compositions and stabilizers, are also referred to as formulation aids within the scope of the invention.

F) Modes of Formulation

The choline ascorbate formulations of the invention can be prepared starting from solid, i.e. crystalline or amorphous, choline ascorbate, liquid choline ascorbate forms such as solutions, dispersions, suspensions or emulsions, or starting from choline ascorbate melts. Choline ascorbate need not be in pure form for this purpose, but may be used in a mixture with other substances which can be used according to the invention, such as stabilizers or processing aids. For the sake of simplicity, reference will be made in the following sections only to choline ascorbate, but this should by no means be interpreted as restrictive.

Various modes of formulation will now be described in detail. Deviations therefrom are, of course, conceivable and can easily be carried out by the skilled worker on the basis of the present invention. He may in this connection also have recourse to comprehensive specialist literature such as, for example, Mollet, Formulierungstechnik, Verlag Wiley-VCH, Weinheim or Heinze, Handbuch der Agglomerationstechnik, Verlag Wiley-VCH, Weinheim; or Hager's Handbuch der Pharmazeutischen Praxis, Springer-Verlag, Heidelberg.

Unless stated otherwise, the formulation processes described below can be applied not only to choline ascorbate in pure form but also to mixtures of choline ascorbate with other active substances and/or to additives which are employed for example for stabilizing the formulation, for regulating the bioavailability, or for changing the color thereof, to mention only a few examples.

1. Encapsulations Starting from Choline Ascorbate Crystals

1.1 Crystals Introduced into a Fluidized Bed or a Mixer with Simultaneous/Subsequent Coating of the Crystals

Encapsulations can be carried out inter alia in mixers or fluidized beds.

i) Description of Mixers:

Mixers operating discontinuously or continuously are preferably employed for this purpose. The active ingredient (i.e. choline ascorbate) is introduced where appropriate together with additives such as, for example, carrier material. Plowshares, paddles, screws or the like ensure more or less vigorous mixing of the product. Conventional examples are plowshare mixers, orbiting screw mixers or similar equipment. It is also possible to employ very shallow, box- or trough-shaped designs having one or more screws. Further designs are high-speed mixers such as the Turbolizer® Mixer/Coater from Hosokawa Micron B.V., and all types of drum coaters. An alternative possibility is mixing of the products by movement of the entire container. Examples thereof are tumbling mixers, drum mixers or the like. A further possibility is to use pneumatic mixers (see Ullmann's Encyclopedia of Industrial Chemistry, Sixth Edition, Mixing of Solids).

The application of coatings or coverings (in the widest sense coating, polymers, waxes, oils, fats, fatty acids etc.) serves to protect the active ingredient, and to delay or speed up the release of active ingredient, to enhance the mechanism of action or to achieve additive effects. It is necessary in some cases on application of the coverings or immediately thereafter to add dusting agents such as talc, silicates or the like, to avoid agglomerates.

The metering/addition of the coating material takes place, where appropriate together with additives, usually through devices for dropwise application or spray application. Examples thereof are injectors, spray heads, single-fluid or multifluid nozzles, in rare cases rotating dripping or atomizing devices. In the simplest case, local addition as concentrated stream is also possible. An alternative possibility is first to introduce the coating material into the mixer and then to add the active ingredient. A further possibility is to add initially solid coating material which melts as a result of wall heating or because of mechanical energy input and covers the active ingredient.

The addition of the coating or encasing materials takes place under superatmospheric, atmospheric or subatmospheric pressure, preferably under atmospheric and subatmospheric pressure.

It is advantageous in some cases to preheat or cool active ingredient and/or encasing material (change in viscosity, change in the wetting properties, influence on solidification properties), and feed in or withdraw heat via the container wall and/or the mixing implements. It is necessary in some cases to remove water or solvent vapors.

To improve the coating properties, it may be beneficial for the mixer to be evacuated and, where appropriate, blanketed with protective gas, such as, for example, nitrogen or noble gas. This should be repeated several times depending on the carrier material.

The addition of active ingredients and encasing materials preferably takes place at different sites in the mixer.

ii) Description of Fluidized Bed:

Preparation can take place discontinuously or continuously in fluidized beds. The particles are agitated by the fluidizing gas which may be hot or cooled as required. Suitable as fluidizing gas is, for example, air or else inert gas (usually nitrogen, but also other conventional inert gases). It is worthwhile in some cases to feed in or withdraw heat via the container wall and via heat exchanger surfaces immersed in the fluidized bed. Suitable fluidized beds, and the necessary peripherals, are known in the art.

Internals assisting a defined agitation of the product often have beneficial effects. Examples thereof are rotating displacers or so-called Wurster pipes and the like.

Discontinuous or continuous metering and, where appropriate, preheating of the active ingredients and additives can take place with the aid of the devices described above, which are known to the skilled worker.

Coated active ingredients can in some cases advantageously be prepared in a combination of mixer and fluidized bed. The reasons for such a combination are likewise state of the art and are known to the skilled worker.

For example, crude granules containing choline ascorbate crystals and prepared in a conventional way can be introduced into a fluidized bed. The latter is fluidized and coated by spraying on an aqueous or nonaqueous, preferably aqueous, solution or dispersion of an organic polymer. The fluid used for this purpose is preferably maximally concentrated but still sprayable, such as, for example, a 10 to 50% by weight aqueous or nonaqueous solution or dispersion of at least one polymer which is selected from polymers of groups a) to f), i) and j) described above.

In another preferred process variant, a 10 to 40% by weight, preferably about 20 to 35% by weight, sprayable aqueous or nonaqueous solution or dispersion of at least one polymer which is selected from polymers of groups g) and h) described above is used for the coating.

Aqueous solutions or aqueous dispersions will in general be preferred for the following reasons: no special measures for working up or recovering the solvents are necessary; no special measures for preventing explosions are necessary; some coating materials are preferably supplied as aqueous solutions or dispersions.

However, in special cases, it may also be advantageous to use a nonaqueous solution or dispersion. The coating material dissolves very well, or an advantageously larger amount of the coating material can be dispersed. It is possible in this way to spray a spray liquid with a higher solids content, leading to shorter process times. The lower enthalpy of vaporization of the nonaqueous solvent likewise leads to shorter process times.

It is particularly preferred to apply coating materials which are physiologically tolerated and contain no water and no solvents and thus can be applied for example as melt. Examples thereof are the abovementioned fats, waxes, fatty acids etc., which may, of course, contain additions where necessary. Suitable additions are, in particular, surface-active substances such as emulsifiers, which have a beneficial effect on the spreading properties of the coating material on the choline ascorbate. Combinations of coating materials which can be sprayed on together or successively are known to the skilled worker. The same applies to influencing the quality of coating by changing the process parameters such as spraying pressure, concentration or viscosity of the liquid, spraying time, pauses between sprayings for solidification, or heat treatments.

Dispersions which can be used according to the invention are obtained by dispersing the above polymers in an aqueous or nonaqueous, preferably aqueous, liquid phase, where appropriate also using a conventional dispersing aid, or preparing the abovementioned waxes or fats as melt. A polymer solution, melt or dispersion is preferably sprayed on by charging a fluidized bed apparatus with the choline ascorbate in solid form (crystals, amorphous solid, where appropriate mixed with auxiliaries or carrier, preferably as crude granules) and, while simultaneously heating the latter, spraying on the spray material. Energy is supplied to the fluidized bed apparatus by contact with heated drying gas, frequently air. Preheating of the solution or dispersion may be worthwhile if this makes it possible for spray material with a higher dry matter content to be sprayed or for the viscosity to be reduced. When organic liquid phases are used, solvent recovery is expedient. The product temperature during the coating can be in the range of about 35 to 50° C. The coating can in principle be carried out in the fluidized bed apparatus in a bottom spray process (nozzle sited in the base inflow plate and sprays upward), in a top spray process (coating is sprayed into the fluidized bed from above) or from the side.

In a second preferred embodiment of the process of the invention for fluidized bed coating, the crude product is introduced into a fluidized bed and powder-coated. The powder-coating is preferably carried out with a powder of a solid polymer which is selected from hydroxypropylmethylcelluloses (HPMC) with a number average molecular weight of about 6000 to 80 000; mixed with a plasticizer. Also suitable for powder-coating are all other coating materials able to exist in powder form and unable to be applied either as melt or as highly concentrated solution (the case with, for example, HPMC, hydroxypropylmethylcellulose).

The powder-coating is preferably carried out by continuously metering the coating material into the crude product present in the fluidized bed. The fine particles of the coating material (particle size in the range from about 10 to 100 μm) become attached to the relatively rough surface of the crude granules. Spraying in a plasticizer solution causes the coating material particles to stick together. Examples of suitable plasticizers are polyethylene glycol solutions, triethyl citrate, sorbitol solutions, liquid paraffin and the like. The coating takes place with gentle heating in order to remove the solvent. The product temperature may in this case be less than about 60° C., such as, for example, about 40 to 50° C.

It is also possible in principle for the powder-coating to be carried out in a mixer. In this case, the powder mixture is metered in, and the plasticizer is sprayed in likewise through a nozzle. Drying takes place by supplying energy through the wall of the mixer and, where appropriate, via the agitator implements. Low product temperatures should be maintained in this case too, as in the coating and drying, in the fluidized bed.

In a third preferred embodiment of the process of the invention, coating of the crude product introduced into a fluidized bed or mixer takes place by means of a melt. The melt in this case preferably comprises at least one polymer selected from
polyalkylene glycols, in particular polyethylene glycols, having a number average molecular weight of about 1000 to 15 000, such as, for example, about 1000 to 10 000; and
polyalkylene oxide polymers or copolymers having a number average molecular weight of about 4000 to 20 000, in particular block copolymers of polyoxyethylene and polyoxypropylene.

Melt-coating in a fluidized bed is preferably carried out by introducing the crude product to be coated into the fluidized bed apparatus. The coating material is melted in an external reservoir and pumped for example through a heatable line to the spray nozzle. It is expedient to heat the nozzle gas. The spraying rate and inlet temperature of the melt must be adjusted so that the coating material still runs satisfactorily on the surface of the granules and covers the latter uniformly. Preheating of the granules is possible before the melts are sprayed in. Melt-coating can also be carried out in principle by a bottom spray process or a top spray process. Melt-coating in a mixer can be carried out in two different ways. Either the crude product to be coated is introduced into a suitable mixer, and a melt of the coating material is sprayed into the mixer. Another possibility is to mix the coating material in solid form with the product. Supplying energy through the container wall or via the mixing implements causes the coating material to melt and thus cover the crude product. It is possible as required to add some release agent from time to time. Examples of suitable release agents are silica, talc, stearates and tricalcium phosphate.

It is possible where appropriate to add other additions such as, for example, microcrystalline cellulose, talc and kaolin to the polymer solution, dispersion or melt used for the coating.

The weight of the coating as a proportion of the total weight of the coated product is in the range from about 1 to 85% by weight, preferably 3 to 50% by weight or 5 to 40% by weight, based on the total weight of the finished product. The residual moisture content of the polymer-coated product is primarily determined by the hygroscopicity of the polymer material. The residual moisture content is generally in the range from about 1 to 10% by weight such as, for example, 1 to 5% by weight, based on the total weight of the coated product.

1.2 Suspension of Choline Ascorbate Crystals in Melts with Subsequent Atomization/Dispersion and Solidification of the Melts

A further alternative is suspension of the choline ascorbate crystals (produced by crystallization, precipitation, drying under atmospheric pressure in vacuo) or of amorphous choline ascorbate in melts of fats, oils, waxes, lipids, lipid-like and lipid-soluble substances with a melting point below the melting point of choline ascorbate. These suspensions are subsequently atomized in a stream of cold gas—with and without use of dusting agents—to result in covered choline ascorbate powder.

The melts are preferably prepared in a first step before the choline ascorbate crystals are added and suspended. The suspension can take place batchwise in a stirred vessel or else continuously, e.g. in pumps suitable for this purpose, or, if the turbulence is sufficiently high, simply in injectors and pipelines. Less preferred, but not precluded, is the use of static mixers. The measures for protective heating, which is necessary where appropriate, of the necessary parts of the system—including the lines and atomizing units—are known to the skilled worker.

Air and nitrogen are suitable and preferred as cooling gas. The gas flow can be cocurrent, countercurrent or crossflow. The process can be carried out in conventional spraying, prilling towers or other containers. Fluidized beds with and without holdup (charged material) are likewise suitable. The process can be operated discontinuously or continuously. The solid can be removed for example in cyclones or filters. Alternatively, it is conceivable for the solid to be collected, with and without after-cooling, in fluidized beds or mixers.

Suitable atomizing units are nozzles (single and twin fluid nozzles or special designs) and atomizing wheels or atomizing disks or atomizing baskets—or special designs thereof.

A further alternative is the dispersion and solidification of these hydrophobic melts in liquids, preferably in liquids in which choline ascorbate and the encasing material are of low solubility. Examples of such liquids are, for example, liquid nitrogen, ethanol, isopropanol, butanol, acetone and dichloromethane. A conventional solid/liquid separation with subsequent drying then lead to the desired dry powder.

1.3 Dispersion of the Crystals in a Lipophilic Environment and Emulsification of These Crystals/Oil Droplets in Aqueous Protective Colloid/Sugar Phase with Subsequent Spray Formulation

Very fine-particle choline ascorbate (produced by precipitation, crystallization, spray drying or grinding) is initially dispersed with and without addition of emulsifiers/stabilizers in a lipophilic environment (such as, for example, melts of fats, oils, waxes, lipids, lipid-like and lipid-soluble substances with a melting point below the melting point of choline ascorbate—all referred to as oil hereinafter). These oil droplets containing the crystalline solid are emulsified in a further process step in an aqueous protective colloid/sugar phase and subsequently spray formulated.

Concerning the preparation and composition of the protective colloid/sugar mixture and the procedure for spray formulation, reference is made to the following section 2.2.

1.4 Encapsulation by Coacervation

Encapsulation of suspended choline ascorbate particles is possible by means of the coacervation process. This process is carried out by using a dispersion liquid which comprises the coating material in dissolved or colloidal form and choline ascorbate solid particles. Reducing the solubility of the coating material induces encapsulation of the choline ascorbate particle. The coacervation technique is described for example in Voigt, Lehrbuch der pharmazeutischen Technologie, Verlag Chemie, chapter 12.4, which is expressly incorporated herein by reference.

2. Encapsulations Starting from an Aqueous Solution

2.1 Spray Formulation of a Protective Colloid/Sugar/Choline Ascorbate/Water Mixture which Optionally Contains Additives (Such as Antioxidants, Salts)

The process is carried out in accordance with EP-A-0 074 050 or DE-A-101 58 046.0 of BASF AG, which is expressly incorporated herein by reference.

Spray-formulated products are prepared by, in a first step, preparing an aqueous solution of a protective colloid, preferably gelatin and/or gelatin derivatives and/or gelatin substitutes, such as pectins and gum arabic, with addition of one or more substances from the group mono-, di- or polysaccharides, where necessary also with addition of corn starch. Then, addition of choline ascorbate, e.g. as crystalline solid (which will then dissolve completely or partially) or as aqueous solution, and where appropriate other additives, such as, for example, hydrophilic or hydrophobic stabilizers or antioxidants, with stirring results in a dispersion, with the aqueous solution of the colloid representing the homogeneous phase of the dispersion. This dispersion is subsequently spray formulated.

Examples of spraying aids which can be employed are a hydrophobic silica, corn starch or metal salts of higher fatty acids. It is also conceivable to use modified corn starch, talc, hydrophilic silica, tricalcium phosphate and calcium silicates or mixtures of two or more of these substances. It is likewise possible to use mixtures of said fatty acids and silicas for the process. Suitable metal salts of higher fatty acids having 16 to 18 C atoms are, for example, calcium or magnesium stearate.

Suitable colloids are preferably animal proteins such as gelatin, for example from 50 to 250 Bloom, or casein. The amount of colloid used is ordinarily 5 to 50% by weight based on the final product with water contents of from 30 to 70% by weight in the dispersion. An alternative possibility is to employ other protective colloids (selected from the examples detailed above).

The spraying aid can be introduced into the spraying chamber in an amount which is from 0.01 to 0.25 times the weight based on the dispersion, above the fluidized bed with uniform dispersion. The spraying aids are introduced directly into the spraying zone. The layer of spraying aid produced during the spraying stabilizes the particles to such an extent that coalescence of the particles on contact in the unsolidified state is prevented. This makes it possible to carry out direct drying in a downstream fluidized bed dryer.

The design of the atomizing unit has no crucial influence on the product. It is possible to employ here for example apparatuses like those described in EP-A-0 074 050.

The spray-formulated products can be prepared in one process variant by spraying the dispersion in a spray tower also using a spraying aid, and collecting the sprayed particles in a fluidized bed. The spraying aid introduced into the spraying chamber in this case is a hydrophobic silica or the metal salt of a higher fatty acid, e.g. having 16 to 18 C atoms, or mixtures with hydrophobic silica, in an amount which is from 0.02 to 0.15 times the weight based on the dispersion (with negligible amounts of other conventional spraying aids such as starch powder being present) above the fluidized bed with uniform dispersion, in particular at temperatures at which no solidification of the colloid of the sprayed particles, which forms a gel where appropriate, yet occurs. The particles which are loaded with the spraying aid and whose colloidal mass has essentially not formed a gel are collected in a fluidized bed, and the particles are dried in the fluidized bed in a manner known per se.

The design of the atomizing unit has no crucial effect on the product. Thus, for example, it is possible to use nozzles or rapidly rotating atomizing disks. The temperature of the dispersion to be atomized is not a critical variable either. It is normally from 30 to 90° C., affording viscosities of from 50 to 1200 mPas with the colloids mentioned. The crucial factor is that at the time of spraying the particles come into contact with the hydrophobic spraying aid, which is introduced in finely divided form directly into the spraying zone.

The great advantage of the process is that the temperature in the spraying chamber need no longer be so low that the active ingredient dispersion forms a gel, or that it is no longer necessary to remove, by large amounts of auxiliary powder, sufficient water for solidification of the droplets to take place. The process makes it possible for example for active ingredient dispersions which no longer solidify even at refrigerator temperatures (+4° C.) to be sprayed at temperatures of from 25 to 30° C. The amounts of the spraying aid for this are in this case only 0.02 to 0.15 times the dispersion.

The spray-formulated product can, in a further process variant, be prepared by spray cooling. This entails a dispersion containing a protective colloid preferably being sprayed by means of an atomizing nozzle or an atomizing wheel at a temperature which is above the gel point of the emulsion, e.g. 30° C. to 90° C., and at a viscosity preferably between 50 and 600 mPas, into a spraying chamber in which the temperature is between 0° C. and 40° C., resulting in microcapsules.

A spraying aid such as, for example, corn starch or modified corn starch where appropriate mixed with other spraying aids can be blown into the spraying chamber in order to prevent agglomeration of the gelatinized microcapsules and adhesion to the walls of the chamber. The spraying aid is preferably added in an amount of from 5 to 50%, measured by the weight of the final product.

The microcapsules can then be transferred into a fluidized bed in which they can, if required, be dried to a residual water content of between 0 and 10% (preferably between 2 and 5%) and in which excess spraying aid is removed. The temperature of the drying air is preferably between about 0° C. and about 100° C.

A particularly preferred variant is spray formulation of a highly concentrated solution of choline ascorbate according to a process described above. For this purpose, firstly a solution of choline ascorbate comprising from 40 to 99% by weight of choline ascorbate in solvent such as, for example, water, preferably 60 to 99% by weight, particularly preferably 80 to 95% by weight of choline ascrobate in water, is prepared. It is possible by adjusting the solution temperature to reach the viscosities suitable for atomization. For example, it is possible to obtain an aqueous solution with a solids content of 95% by weight and a viscosity of less than 1000 mPas at temperatures of 60° C. Where necessary, stabilizing additions are added to this solution. The solution obtained in this way can then be spray formulated with the aid of a single-fluid nozzle at elevated pressure (e.g. between 3 and 300 bar) with simultaneous use of dusting agents such as hydrophobic silica (e.g. Sipernat D17 from Degussa) or modified corn starch. The particles obtained in this way are collected and dried for example in the fluidized beds described above or else in vacuum apparatuses and, where appropriate, subsequently coated with a protective layer, for example as described above.

It is conceivable furthermore to prepare, based on R. A. Morten: Fat-Soluble Vitamins, Pergamon Press, 1970, pages 131 to 145, dispersions/emulsions of solid choline ascorbate and then to prepare therefrom, as described, powders of the invention.

2.2 Preparation of Spray Granules or a Spray Agglomerate with Subsequent Coating

In this process, an aqueous choline ascorbate solution is added to a fluidized bed and converted into a solid powder. The fluidized bed can again be operated discontinuously or continuously. The aqueous choline ascorbate solution is preferably sprayed onto a receiver in the fluidized bed. The receiver may be choline ascorbate itself or a carrier material. It is likewise possible to start up without receiver. The solution can again be sprayed in a top spray or bottom spray mode. Atomizing units inserted laterally in the container wall are also possible. It may be advantageous to adapt the distance of the atomizing units from the fluidized solid in accordance with the properties of the solid (e.g. granulation tendency). The atomizing units preferably employed are atomizing nozzles (pressure nozzles such as single-fluid nozzles, twin-fluid nozzles or special designs). The process can be operated with and without dust recycling.

The skilled worker is able to exert a beneficial influence on the properties of the solid through the setting of the process parameters and through correct choice of additives. Thus, for example, it is possible to produce complex granules with high particle and bulk density and agglomerates with excellent reconstitution and/or tableting properties.

The desired particle size of the final product can be adjusted within wide limits. The average particle size can be between 20 μm and 5000 μm. It is preferably between 50 μm and 2000 μm and particularly preferably between 150 μm and 600 μm.

If the desired average particle size is, for example, about 400 μm, it may be beneficial to start with an average particle size of the receiver material of about 30 to 50 μm. The receiver material can be produced for example by previous grinding of coarse choline ascorbate or inert carrier material or for example by spray drying in the same or in a different apparatus suitable for this purpose. In some circumstances the receiver material also results as material cleaned off filters or cyclones or other solid separators, or may be processed in a suitable particle size resulting from other processes.

The use of special receiver material can be dispensed with in continuous processes, but also in discontinuous processes, if the parameters are chosen suitably.

It is then possible to produce the desired agglomerates for granules by spraying on the aqueous choline ascorbate solution or else by spraying on binder liquid alone.

It is, of course, possible to coat the produced solid powders with a protective coat in the same or in a different apparatus.

A particularly interesting variant consists in adding to the aqueous choline ascorbate solution additives which have a beneficial effect firstly on the crystal structure (crystalline or amorphous) and on the undesired tendency to discoloration of the choline ascorbate.

Additives for influencing the crystal structure (size and/or shape) are known. These are multiply charged ions, organic molecules or surfactants. A distinction is made between so-called tailored and multifunctional additives. Tailored additives have great similarity to the constituents of the crystal. They are adsorbed onto the growth surfaces, retard growth there and thus enlarge this surface. Proportions of up to 10% of auxiliaries are necessary for complete inhibition of growth. Multifunctional additives are employed more often than tailored ones, especially for inorganic crystals. In most cases, polyphosphonates or polycarboxylates, such as, for example, polyacrylates, are employed. These polyelectrolytes wet the growth surfaces and block growth there. Amounts in the ppm range are often sufficient.

It is also possible in principle to replace water partly or completely by organic solvents.

It is also conceivable to carry out the described process in other apparatuses such as, for example, mixers.

In a further variant, the abovementioned protective colloids, sugars, emulsifiers, stabilizers etc. can be added to the solution before spraying in, or introduced separately through an alternative atomizing unit.

2.3 Formulation of a Choline Ascorbate Solution with a Carrier

A further variant is to add the choline ascorbate solution to a carrier. Porous carrier materials are preferably employed. The mixers and fluidized beds described above in section 1.1 are suitable as devices for preparing these formulations.

The carriers normally used are inert materials. An “inert” carrier must not show any adverse interactions with the components employed in the formulation of the invention and must be acceptable for use as auxiliary in the respective uses, e.g. in human foods, human food supplements, animal foods, animal food additives, pharmaceutical and cosmetic preparations.

Examples which may be mentioned of suitable carrier materials are: low molecular weight inorganic or organic compounds, and high molecular weight organic compounds of natural or synthetic origin. Examples of suitable low molecular weight inorganic carriers are salts such as sodium chloride, calcium carbonate, sodium sulfate and magnesium sulfate or kieselguhr or silicas such as silicon dioxides or silica gels or silica derivatives such as, for example, silicates.

Examples of suitable organic carriers are, in particular, sugars such as, for example, glucose, fructose, sucrose, dextrins, starch products, especially corn starch and cellulose products. Examples which should be mentioned of other organic carriers are: corncob meal, ground rice husks, wheat bran or cereals flours such as, for example, wheat, rye, barley and oatmeal or bran or mixtures thereof.

Examples of preferred porous carriers are silicas such as, for example, the Sipernat products from Degussa or the Tixosil products from Rhodia, Lyon.

The carrier material may be present in the formulation of the invention in a proportion of about 10 to 85% by weight, preferably about 20 to 85% by weight, on a dry basis.

2.4 Preparation of Granules or Extrudates

For this purpose, firstly wet granules are prepared for example from a choline ascorbate solution, carriers (such as, for example, corn starch or microcrystalline cellulose) and binders (such as, for example, HPMC, HPC or HMC) in a mixer. These wet granules are then shaped in a further process step in an extruder (meat grinder, basket extruder, twin screw extruder, etc.), where appropriate after-treated (compaction, rounding, etc.), dried (e.g. again in a fluidized bed or in a contact dryer), and, if necessary, coated again. Suitable apparatuses are, for example, those of the NICA System® from Aeromatic-Fielder.

Granules can also be prepared by introducing carriers and, where appropriate, additives into a mixer and, after addition of solid choline ascorbate and binder (preferably binder liquid—in the simplest case water), producing compact granules.

The mixer is preferably a paddle mixer or plowshare mixer. The liquid components are added (applied dropwise or sprayed on) to result in a pasty, tacky phase. The pasty phase is dispersed through suitable choice of the speed of rotation of the mixing implements and/or high-speed knives, to result in compact granules. Very large lumps are dispersed by mixing implements and knives. On the other hand, fine powders are agglomerated thereby.

The mode of operation is discontinuous or continuous. It is often necessary to supply or remove heat via a heating jacket. The crucial step is the combination of binder liquid, mechanical energy input by mixing implements and knives and establishment of the necessary granulation time

A coating can be applied subsequently in the mixer with a lower speed of rotation of the mixing implements and stationary knives or in a downstream mixer of related construction.

The shaping can also take place by forcing the pasty, tacky phase through the die of an extruder. The process results in extrudates which are, where appropriate, subsequently dried and then coated.

2.5 Emulsification of a Choline Ascorbate Solution in Wax with Subsequent Shaping

In analogy to section 1.2, but starting from the aqueous, aqueous-organic or organic choline ascorbate solution, in a first step with and without addition of auxiliaries (emulsifiers, stabilizers) firstly an emulsion of choline ascorbate in melts of fats, oils, waxes, lipids, lipid-like and lipid-soluble substances is prepared. The subsequent shaping again takes place in a stream of cold gas as in section 1.2.

3. Encapsulations Starting from a Melt

3.1 Preparation of a Choline Ascorbate/Wax/Fat Dispersion with Subsequent Atomization/Dispersion and Solidification

An anhydrous melt of choline ascorbate is dispersed with the addition of aids for example in melts of fats, oils, waxes, lipids, lipid-like and lipid-soluble substances with a melting point above or below the melting point of choline ascorbate. These dispersions are subsequently atomized in a steam of cold gas—with and without use of dusting agents—so that covered choline ascorbate powder is produced. Concerning the subsequent procedure, reference may be made to the statements in section 1.2.

3.2. Preparation of Choline Ascorbate Solids in a Vacuum Apparatus and, where Appropriate, Subsequent Granulation/Agglomeration/Compaction and, where Appropriate, with Subsequent Coating.

In this process, an aqueous solution of choline ascorbate or a solution of choline ascorbate in aqueous-organic or organic solvents is evaporated to a solid in a vacuum apparatus, where appropriate with use of carriers and additives. It is possible in the same apparatus or in a different apparatus for the solid to be, where appropriate with addition of binders, agglomerated, granulated, compacted and, where necessary, again comminuted, classified and, where appropriate, coated with a protective layer.

Suitable apparatuses are, for example, conventional vacuum dryers like those known to the skilled worker. The choline ascorbate can be introduced as solution or else as solid. The wall temperature is preferably not above the melting point of choline ascorbate, because decomposition must be expected at higher temperatures. The process is preferably carried out at a pressure in the range between atmospheric pressure and technically possible subatmospheric pressure, particularly preferably under a pressure between 0 and 500 mbar, absolute. A preferred variant is the use of inert gas such as, for example, nitrogen as stripping gas in order to minimize the partial pressures of oxygen and water vapor in the vacuum apparatus. Where necessary, the required particle size is adjusted in the vacuum apparatus by addition of binder liquid, or compaction, agglomeration or granulation and, where necessary, coating of the resulting particles are carried out in downstream apparatuses.

3.3 Spraying and Solidification of a Melt in the Presence of a Dusting Agent which is Incorporated and, If Appropriate, Assumes the Function of a Coating

A melt of choline ascorbate is atomized where appropriate with the addition of additives in a stream of cold gas—with and without use of dusting agents—to result in encased choline ascorbate powder. The dusting agents (cf. statements above) are suitable for preventing the coalescence where necessary of drops which have solidified only on the surface. An example of a suitable dusting agent which may be mentioned here is SiO₂.

3.4 Dropwise Application/Spray Application of a Melt to a Porous Carrier

In analogy to section 2.3, but with the difference of starting from a choline ascorbate melt in place of a solution, emulsion or suspension, choline ascorbate is added to a, preferably porous, support and further processed.

3.5 Preparation of Granules/Extrudates

In analogy to the procedure described in section 2.4, but with use of a choline ascorbate melt, corresponding granules/extrudates are prepared.

G) Applications of Choline Ascorbrate Formulations of the Invention

Choline ascorbate formulations of the invention are used just like conventional choline products as addition to human and animal foods or addition to human and animal food supplements such as, for example, multivitamin products. The formulation stabilized according to the invention can for this purpose be incorporated in the desired amount and in a manner known per se to conventional human and animal foods and human and animal food supplements.

The choline ascorbate formulations of the invention are additionally suitable for preparing pharmaceuticals such as, in particular, products for the treatment and/or prevention of cirrhosis of the liver or other liver disorders. Further potential areas of application to be mentioned are: improvement of cognitive functions; treatment and/or prevention of various types of dementia or Alzheimer's disease; and other neurodegenerative disorders; and reduction of plasma homocysteine levels and the prevention, associated therewith, of cardiovascular disorders.

Food supplements can likewise be used for the purpose of the invention.

The pharmaceutical compositions of the invention for treating an individual, preferably a mammal, in particular a human, agricultural or domestic animal can be prepared in a manner known per se. Thus, the stabilized choline ascorbate is usually administered in the form of pharmaceutical compositions which comprise a pharmaceutically acceptable excipient with at least one choline ascorbate formulation of the invention and, where appropriate, other active ingredients. These compositions can be administered for example by oral, rectal, transdermal, sublingual, buccal, subcutaneous, intravenous, intramuscular or intranasal route.

Examples of suitable pharmaceutical formulations are solid drug forms such as oral powders, dusting powders, granules, tablets, pastilles, sachets, cachets, sugar-coated tablets, film-coated tablets, capsules such as hard and soft gelatin capsules, suppositories or vaginal drug forms; semisolid drug forms such as ointments, creams, hydrogels, pastes or plasters; and liquid drug forms such as solutions, emulsions, especially oil-in-water emulsions, suspensions, for example lotions, preparations for injection and infusion, eyedrops and eardrops. Implanted delivery devices can also be used to administer formulations of the invention. It is also possible to use liposomes, microspheres or polymer matrices.

In the preparation of the compositions, choline ascorbate formulations of the invention are usually mixed or diluted with an excipient. Excipients may be solid, semisolid or liquid materials which serve as vehicle, carrier or medium for the active substance.

Examples of suitable excipients include lactose, dextrose, sucrose, sorbitol, mannitol, starches, gum acacia, calcium phosphate, alginates, tragacanth, gelatin, colloidal anhydrous silica, calcium silicate, microcrystalline cellulose, polyvinylpyrrolidone and derivatives thereof, cellulose and derivatives thereof, water, alcohol/water mixtures, syrup and methylcellulose. The formulations may additionally comprise pharmaceutically acceptable carriers or conventional excipients such as lubricants, for example talc, magnesium stearate, oils of vegetable origin and mineral oil; wetting agents, emulsifying and suspending agents; preserving agents such as methyl and propyl hydroxybenzoates; antioxidants; antiirritants, chelating agents; tablet coating aids; emulsion stabilizers; film formers; gel formers; odor-masking agents; masking flavors; resins; hydrocolloids; solvents; solubilizers; neutralizers; permeation promoters; pigments; quaternary ammonium compounds; refatting and superfatting agents; ointment, cream or oil bases; silicone derivatives; spreading aids; stabilizers; sterilants; suppository bases; tablet excipients such as binders, fillers, lubricants, disintegrants or coatings; propellants; desiccants; opacifying agents; flow regulators, thickeners; waxes; plasticizers; white oils. An arrangement concerning this is based on specialist knowledge as described. for example, in Fiedler, H. P., Lexikon der Hilfsstoffe für Pharmazie, Kosmetik und angrenzende Gebiete, 4th edition, Aulendorf: ECV-Editio-Cantor-Verlag, 1996, or Hager's Handbuch der Pharmazeutischen Praxis, Springer Verlag, Heidelberg. The excipients can be employed singly or in a mixture.

The present invention is now explained in more detail with reference to the following exemplary embodiments.

General Information:

Unless stated otherwise, choline ascorbate (CAS) having a melting point of 120 to 130° C. is employed in the following experiments.

The choline ascorbate crystals employed in the formulation examples were obtained by crystallization by cooling a methanolic solution, with subsequent solid/liquid separation and drying.

The CAS solution employed in the examples was an aqueous solution in each case. The composition of the solution was usually 40% water and 60% choline ascorbate. The solution was in each case prepared from the abovementioned choline ascorbate crystals by adding water. Where necessary, additives were added to the solution to avert the tendency to discoloration.

In those examples starting from a CAS melt, the abovementioned choline ascorbate crystals were warmed, where appropriate mixed with a stabilizer as defined above, and heated above their melting point.

### EXAMPLE 1

Determination of the Stability of Choline Compounds in Solution

Solid choline ascorbate is firstly prepared in a manner known per se as disclosed in DE-A-101 090 73. 0.2 mol of ascorbic acid was added to 0.2 ml of trimethylamine in methanol (25% by weight) while cooling to 0° C. 0.2 ml of ethylene oxide was passed into this mixture in such a way that the reaction temperature did not exceed 0-5° C. After the end of the reaction, the reactor was flushed with nitrogen and stirred further at a temperature between 0 and 5° C. The choline ascorbate which had formed was crystallized from the reaction mixture, filtered off, washed with methanol and recrystallized from methanol and used for further purification. Colorless crystals with a melting point between 123.5° and 124.4° C. were obtained in a yield of 80%. The crystals were characterized as choline ascorbate (anhydrous) by means of elemental analysis, ¹³C-NMR spectroscopy and single crystal structural analysis.

A 50% strength solution (in 1:1 water/methanol) of this choline ascorbate (melting point 123-124° C.) is stirred at reflux (65° C.) in an air atmosphere for several hours. At the start of the experiment and after various reaction times the degree of discoloration is determined by means of the Gardner (DIN-ISO 4630) or Hazen (DIN-ISO 6271) color number.

In analogous way, 50% strength aqueous/methanolic solutions of L(+)-ascorbic acid, sodium ascorbate and choline bitartrate were investigated. The results are summarized in the following Table 1.

TABLE 1

| | |
|---|---|
| Reaction time | Color number |

| Substance | [h] | Gardner | Hazen |
|---|---|---|---|
| Choline ascorbate | 0 | 0.1 | 28 |
| Without additive | 1 | 1.9 | 305 |
| | 2 | 3.5 | 758 |
| | 7 | 6.3 | >1000 |
| L-(+)-Ascorbic acid | 0 | 0.1 | 27 |
| Without additive | 1 | 0.1 | 31 |
| | 7 | 0.1 | 29 |
| Sodium ascorbate | 0 | 0.1 | 26 |
| Without additive | 1 | 0.1 | 28 |
| | 7 | 0.1 | 31 |
| Choline bitartrate | 0 | 0.1 | 27 |
| Without additive | 1 | 0.1 | 30 |
| | 7 | 0.1 | 28 |

The results of the experiment clearly reveal the surprisingly great instability of unstabilized choline ascorbate compared with other choline compounds and ascorbic acid, whose instability was already known.

### EXAMPLE 2

Preparation of a Stabilized Choline Ascorbate Solution

A 50% strength solution (in 1:1 water/methanol) of choline ascorbate (melting point 123-124° C.), prepared as in Example 1, is stirred at reflux (65° C.) without or with one percent by weight of various stabilizing additives in an air atmosphere for several hours. The stabilizing effect of the respective additive is observed via determination of the color number as described in Example 1.

Table 2 below lists, besides the additive and reaction time, the Gardner and Hazen color numbers as a function of time to prove the stabilizing effect of the respective additive.

TABLE 2

| Choline ascorbate stabilized | Reaction time | Color number | |
|---|---|---|---|
| with additive [1% by weight] | [h] | Gardner | Hazen |
| without additive | 0 | 0.1 | 28 |
| (comparative) | 1 | 1.9 | 305 |
| | 2 | 3.5 | 758 |
| | 7 | 6.3 | >1000 |
| Cysteine | 0 | 0.1 | 33 |
| | 1 | 0.1 | 29 |
| | 4 | 0.1 | 35 |
| | 7 | 0.1 | 27 |
| Sodium dithionite | 0 | 0.5 | 90 |
| | 1 | 0.1 | 29 |
| | 7 | 0.1 | 33 |
| Thioglycolic acid | 0 | 0.1 | 28 |
| | 1 | 0.1 | 30 |
| | 7 | 0.1 | 33 |
| Dihydrolipoic acid | 0 | 0.1 | 30 |
| | 1 | 0.2 | 50 |
| | 4 | 0.2 | 53 |
| | 7 | 0.2 | 48 |
| Lipoic acid | 0 | 0.2 | 51 |
| | 1 | 1.0 | 182 |
| | 7 | 4.4 | 960 |
| Glutathione | 0 | 0.1 | 31 |
| | 1 | 0.2 | 63 |
| | 4 | 0.4 | 122 |
| | 7 | 0.6 | 185 |
| N-Acetylcysteine | 0 | 0.1 | 30 |
| | 1 | 0.3 | 72 |
| | 4 | 0.9 | 162 |
| | 7 | 1.7 | 285 |
| Uric acid | 0 | 0.1 | 26 |
| | 1 | 0.3 | 68 |
| | 3 | 0.9 | 162 |
| | 5 | 1.4 | 237 |
| | 7 | 1.9 | 310 |
| Phenylboronic acid | 0 | 0.1 | 31 |
| | 1 | 0.6 | 193 |
| | 4 | 2.7 | 589 |
| | 7 | 4.4 | 975 |
| Hypophosphorous acid | 0 | 0.1 | 30 |
| | 1 | 0.2 | 58 |
| | 4 | 0.6 | 128 |
| | 7 | 1.2 | 274 |
| Phosphorous acid | 0 | 0.1 | 30 |
| | 1 | 0.2 | 122 |
| | 4 | 0.7 | 163 |
| | 7 | 1.4 | 299 |

The data in Table 2 above proves the completely surprising finding according to the invention that choline ascorbate can be stabilized in an advantageous manner despite its extremely great tendency to discoloration by adding small amounts of suitable stabilizers.

### EXAMPLE 3

Preparation of a Solid Stabilized Choline Ascorbate

Choline ascorbate is converted into an aqueous solution, mixed with a stabilizer of the invention and concentrated (vacuum, T=70-80° C.). The stabilized product crystallizes out after cooling.

### EXAMPLE 4

Preparation of a Choline Ascorbate Formulation by Fluidized Bed Coating with a Fat

The product to be coated is a supercooled melt of choline ascorbate and cysteine or a mixture of crystals thereof, in each case comprising 98% by weight CAS and 2% by weight cysteine (with an average particle size of about 300 μm). The coating material used was a fat having a melting point of 60 to 64° C. (Rucawar FH from Aarhus Olie, Denmark).

A Niro-Aeromatic, type MP-1 laboratory fluidized bed was available for carrying out the experiments. The receiver vessel employed was a plastic cone with a base inflow plate diameter of 110 mm and a perforated plate with 8% free area.

The choline ascorbate (500 g) introduced into the fluidized bed was heated while fluidizing with air at a rate of 30 m³/h to a product temperature of 40° C. The fat (125 g) was melted in a glass beaker in an oil bath at 80° C. and sprayed onto the choline ascorbate using a 1.2 mm twin fluid nozzle in a top-spray process by reduced-pressure intake through a heated line at a spraying pressure of 2 bar with spraying gas heated to 85-90° C. During the spraying process, the air rate was increased to 100 m³/h in order to ensure thorough mixing and a uniform coating layer. The spraying time was 5 min, with the product temperature being 40 to 43° C. and the inlet air temperature being about 40-50° C.

### EXAMPLE 5

Formulation Example—Multivitamin Tablet

A multivitamin tablet of the following composition:

| | | |
|---|---|---|
| β-Carotene | 5 | mg |
| Vitamin E | 10 | mg |
| Vitamin C | 60 | mg |
| Vitamin D | 1.2 | mcg |
| Thiamine | 1.4 | mg |
| Riboflavin | 1.6 | mg |
| Pyridoxine HCl | 2.2 | mg |
| Vitamin B₁₂ | 1 | mcg |
| Niacin | 18 | mg |
| Pantothenic acid | 6 | mg |
| Folic acid | 200 | mcg |
| Biotin | 150 | mcg |
| Stabilized choline ascorbate | 150 | mg |
| (prepared as in Example 4) | | |
| Magnesium | 100 | mg |
| Zinc | 15 | mg |
| Manganese | 2.5 | mg |
| Selenium | 62 | mcg |

is prepared in a manner known per se using conventional formulation aids known to the skilled worker.

### EXAMPLE 6

Formulation Example—B-Group Vitamin Tablet

A vitamin tablet of the following composition:

| | | |
|---|---|---|
| Vitamin C | 500 | mg |
| Thiamine | 100 | mg |
| Riboflavin | 100 | mg |
| Vitamin B₆ | 100 | mg |
| Vitamin B₁₂ | 500 | mcg |
| Niacin | 100 | mg |
| Pantothenic acid | 100 | mg |
| Folic acid | 400 | mcg |
| Biotin | 50 | mcg |
| Stabilized choline ascorbate | 500 | mg |
| (prepared as in Example 4) | | |

is prepared in a manner known per se using conventional formulation aids known to the skilled worker.

### EXAMPLES 7a AND 7b

Fluidized-Bed Coating of Choline Ascorbate

Apparatus and procedure as in Example 4. The temperatures and spraying times were adapted. 400 g of choline ascorbate-containing solid were introduced into the cone.

| | | Composition |
|---|---|---|
| Example | | of the final |
| No. | Coating | product |
| 7a | 36.6 g of gelatin (91% DM) and 69.5 g of | 80% choline |
| | lactose (96% DM) were dissolved in 180 g | ascorbate |
| | of drinking water at 60° C. 100 g of | 20% coating |
| | coating material (calculated dry) were | |
| | sprayed on. | |
| | Gelatine 100 Bloom Avon DFG | Loss on drying |
| | | <0.8% |
| 7b | 100 g of polyethylene glycol (PEG) were | 82% choline |
| | dissolved in 100 g of drinking water. | ascorbate |
| | 88 g of coating material (calculated dry) | 18% coating |
| | were sprayed on. | |
| | PEG: Lutrol E 6000 from BASF | Loss on drying |
| | | <0.2% |

DM = dry matter

### EXAMPLES 8a AND 8b

Choline Ascorbate Coating in a Stirred Flask

a) The product to be coated was again choline ascorbate as described in Example 4.

50 g of the solid were introduced into a four-neck reaction flask and heated to 60° C. in an oil bath while stirring.

The coating material used was beef tallow with a melting point of 56-60° C. (Edenor NHTI-G from Henkel/Cognis). The beef tallow was melted at a temperature of 80° C. in a glass beaker. A pipette was used to introduce 12.5 g of the Edenor NHTI-G melt dropwise onto the stirred choline ascorbate in the four-neck flask. The stirring speed was 250-300 rpm. After addition of the melt, the choline ascorbate coated with beef tallow was cooled with stirring, and the melt solidified. Choline ascorbate particles with about 20% coating were obtained.

b) The experiment was repeated with addition of 33.5 g of the Edenor NHTI-G melt to 50 g of choline ascorbate under comparable conditions. Choline ascorbate particles with about 40% coating were obtained.

### EXAMPLES 9a TO 9c

Choline Ascorbate Coating in a Stirred Flask

Apparatus and procedure as in Example 8. The temperatures were adapted to the melting points. 50 g portions of choline ascorbate as described in Example 4 were introduced into the four-neck reaction flask.

| | | Composition |
|---|---|---|
| Example | | of the final |
| No. | Coating | product |
| 9a | 12.5 g of Rucawar FH (hydrogenated | 80% choline |
| | rapeseed oil, contains 30 ppm citric acid) | ascorbate |
| | (Rucawar FH from Aarhus Olie, Denmark) | 20% coating |
| 9b | 12.5 g of Bassao E 63 (hydrogenated shea | 80% choline |
| | nut oil. contains 30 ppm of citric acid) | ascorbate |
| | (Bassao E 63 from Aarhus Olie, Denmark) | 20% coating |
| 9c | 12.5 g of polyethylene glycol | 80% (or 60%) |
| | (PEG Lutrol E 6000) (or 33.3 g of | choline |
| | polyethylene glycol (PEG Lutrol E 6000)) | ascorbate and |
| | (PEG: Lutrol E 6000 from BASF) | 20% (or 40%) |
| | | coating |

### EXAMPLES 10a AND 10b

Spray Granulation of Aqueous Choline Ascorbate Solution in a Fluidized Bed

a) A Niro-Aeromatic, type MP-1 laboratory fluidized bed was available for carrying out the experiments. The receiver vessel employed was a plastic cone with a base inflow plate diameter of 110 mm and a perforated plate with 8% free area.

300 g of choline ascorbate (cf. Example 4) were introduced as receiver material into the cone of the fluidized bed. 300 g of the same solid were dissolved in 129 g of drinking water.

The choline ascorbate (300 g) introduced into the fluidized bed was heated to a product temperature of 47° C. while fluidizing with air at a rate of 30-40 m³/h. The product temperature was measured in the fluidized bed. The aqueous choline ascorbate solution was sprayed in a top-spray process by reduced-pressure intake at a spraying pressure of 1.5 bar using a twin-fluid nozzle (nozzle diameter 1.2 mm). The spraying time was about 35 min, with the product temperature being between 45 and 47° C. and the inlet air temperature being about 58-66° C. The discharged product comprised 568 g of a fine white product. The loss on drying of the product was about 0.6%.

b) The abovementioned experiment was repeated without introducing choline ascorbate into the fluidized bed. For this purpose, 500 g of choline ascorbate (cf. Example 4) were dissolved in 250 g of drinking water. With virtually unchanged operating conditions it was possible within an experimental period of about 160 min to produce about 450 g of a white granulated product. The loss on drying of the product was about 0.5%. Loose thin deposits remained on the wall and filter in the system.

Since spray granulation in a fluidized bed was possible without a receiver, this also demonstrates that conventional spray drying is possible under similar conditions.

### EXAMPLES 11a AND 11b

Spray Granulation of Aqueous Choline Ascorbate Solution in a Fluidized Bed with Addition of Additives

Apparatus and procedure as in Example 10: startup without receiver.

| Example | |
|---|---|
| No. | Experimental parameters |
| 11a | Spraying solution: 475 g of choline ascorbate and |
| | 25 g of dihydrolipoic acid were dissolved in 250 g |
| | of drinking water |
| | Composition of the final product |
| | corresponds to the spraying solution |
| 11b | Spraying solution: 475 g of choline ascorbate and |
| | 25 g of L-cysteine (from Aldrich) were dissolved in |
| | 250 g of drinking water |
| | Composition of the final product |
| | corresponds to the spraying solution |

### EXAMPLE 12

Spray Formulation of Aqueous Choline Ascorbate Solution in a Fluidized Bed

170 g of drinking water were introduced into a glass beaker and 280 g of the choline ascorbate crystals (cf. Example 4) were slowly added and dissolved with stirring. The result was an aqueous solution with a solids content of 62%. This solution was sprayed at a temperature of 60° C. and a spraying pressure of 4 bar using a single-fluid nozzle into a laboratory spray tower. During the spraying, hydrophobic silica (Sipernat D 17®, Degussa) was blown into the spraying zone. A moist powder was obtained and was subsequently predried in a laboratory suction filter and finally dried using a rotary evaporator at a water bath temperature of 50° C. and a pressure of 40 mbar within 5 h.

### EXAMPLE 13

Preparation of a Dissolved Choline Ascorbate Formulation for Determination of the Color Number

A solid CAS formulation is homogenized in a mortar and stirred in a solvent mixture composed of equal parts of water and methanol at room temperature for 15 minutes. The initial weight of formulated product is chosen so that the resulting solution contains about 10% by weight CAS. Any undissolved constituents are removed. The Gardner and/or Hazen color numbers are determined on the resulting solution without delay.

### EXAMPLE 14

Stabilization of Choline Ascorbate or Choline Salt/ascorbic Acid Mixtures by Additives

The effect of stabilizers on choline ascorbate and various choline salt/ascorbic acid mixtures was investigated in the following investigations. The experimental results are compiled in table 3. The experiments took place under the following conditions: 10% strength solutions in water/methanol (1:1)—7 h at 65° C.

TABLE 3

Color number

| | | |
|---|---|---|
| Substance | Gardner | Hazen |

no additive

| | | |
|---|---|---|
| Choline ascorbate | 6.3 | >1 000 |
| L-Ascorbic acid | 0.1 | 27 |
| Sodium ascorbate | 0.1 | 31 |
| Choline chloride | 0.1 | 35 |
| Choline bitartrate | 0.1 | 32 |

Choline mixtures - no additive

| | | |
|---|---|---|
| Ascorbic acid/choline chloride | 4.4 | 975 |
| Sodium ascorbate/choline chloride | 6.1 | >1 000 |
| Ascorbic acid/choline bitartrate | 2.0 | 320 |
| Sodium ascorbate/choline bitartrate | 5.1 | >1 000 |

Choline mixtures with 1% Cys

| | | |
|---|---|---|
| Ascorbic acid/choline chloride | 0.1 | 33 |
| Sodium ascorbate/choline chloride | 0.1 | 45 |
| Ascorbic acid/choline bitartrate | 0.1 | 28 |
| Sodium ascorbate/choline bitartrate | 0.1 | 36 |
| Choline ascorbate | 0.1 | 27 |

As is evident from the color numbers, the colors not only of choline ascorbate but also of mixtures of other choline salts with ascorbic acid are made distinctly more stable by the stabilizer.

### EXAMPLE 15

Determination of the Moisture Stability of a Solid Choline Ascorbate Formulation

A few grams of a solid choline ascorbate formulated according to the invention are put into a glass dish so that the bottom of the dish is uniformly covered with the powdered solid. A desiccator is prepared with an atmosphere defined by a saturated aqueous sodium chloride solution located in the bottom of the desiccator. The gas atmosphere in the desiccator has a relative gas humidity of about 76%. The dish with choline ascorbate is placed in the desiccator and stored at room temperature for 3 days.

After 3 days, the dish with choline ascorbate is removed from the desiccator and assessed. Unformulated choline ascorbate is entirely or partly in the form of a liquid. Choline ascorbate formulated according to the invention is still in the form of a powdered solid after 3 days. No partial or complete deliquescence or dissolving of the formulation is to be observed. The solid may, however, have taken up water during storage and display limited flow properties. On filtration (with or without application of a water pump vacuum) through a suction filter funnel, e.g. D2 (40-100 μm), no liquid phase can be separated from formulations of the invention after standardized storage.

We claim:

1. A solid choline ascorbate formulation with reduced sensitivity to external stress factors, characterized in that a solution of this formulation has under standard conditions a Gardner color number (determined as specified in DIN-ISO 4630 or ASTM D 1544-80) of <4.5, and/or a Hazen color number (determined as specified in DIN-ISO 6271 or ASTM D 1045-68, ASTM D 26349 or ASTM D 1209-69) of <800; and does not deliquesce on storage under standard conditions in moist ambient air.

2. A formulation as claimed in claim 1, wherein
a) choline ascorbate is surface-coated with an inert coating composition;
b) choline ascorbate is embedded in an inert matrix; or
c) a porous carrier is loaded with choline ascorbate, and the loaded carrier is surface-coated where appropriate with an inert coating composition.

3. A formulation as claimed in any of the preceding claims, which additionally comprises an effective amount of at least one addition which further reduces the tendency to discoloration of choline ascorbate.

4. A formulation as claimed in claim 3, wherein the addition which further reduces the tendency to discoloration of choline ascorbate is mixed with the choline ascorbate and/or is present in the surface coating, in the inert matrix or in the porous carrier.

5. A formulation as claimed in claim 3 or 4, wherein the stabilizer is present in a proportion of about 0.05 to 30 mol % based on the molar content of choline ascorbate.

6. A formulation as claimed in any of claims 3 to 4, wherein the stabilizer is selected from sulfur-containing, phosphorus-containing or boron-containing compounds; carboxylic acids and carboxylic acid derivatives; vitamins and vitamin precursors and derivatives; natural product mixtures; hydroxy- or alkoxyaromatic compounds; reductones or mixtures thereof.

7. A formulation as claimed in claim 6, wherein
a. the sulfur-containing stabilizer is selected from cysteine, cystine, N-acetylcysteine, thioglycolate, glutathione, dihydrolipoic acid, lipoic acid, sodium dithionite, methionine and thiourea;
b. the phosphorus-containing stabilizer is selected from phosphorous and hypophosphorous acid;
c. the boron-containing stabilizer is phenylboronic acid;
d. the carboxylic acids and carboxylic acid derivatives are selected from uric, lactic, malic, citric and excess ascorbic acid; and ascorbyl palmitate;
e. the vitamins, vitamin precursors and derivatives are selected from alpha-, beta- and gamma-tocopherol, tocotrienol and more water-soluble vitamin E derivatives; carotenoids; isoflavones; flavonoids and other naturally occurring polyphenols;
f. the natural product mixture is a rosemary extract;
g. the reductone is hydroxyacetone; and
h. the hydroxy- or alkoxy-aromatic compounds are selected from 6-ethoxy-1,2-dihydro-2,2,4-trimethylquinoline (ethoxyquin), t-butylhydroxytoluene and t-butylhydroxyanisole;
or the stabilizer is a functional derivative, having a stabilizing action, of one of the above compounds.

8. A formulation as claimed in any of the preceding claims, wherein the choline ascorbate content is in a range from about 5 to 95% by weight based on the total weight of the formulation.

9. A formulation as claimed in any of the preceding claims, which is coated with a coating composition comprising at least one compound selected from:
a) polyalkylene glycols;
b) polyalkylene oxide polymers or copolymers;
c) substituted polystyrenes, maleic acid derivatives and styrene/maleic acid copolymers;
d) vinyl polymers either alone or in combination with other compounds, such as cellulose ethers or starches;
e) vinylpyrrolidone/vinyl acetate copolymers;
f) polyvinyl alcohols, and polyphthalic acid vinyl esters;
g) hydroxypropylmethylcelluloses;
h) alkyl (meth)acrylate polymers and copolymers;
i) polyvinyl acetates, where appropriate stabilized with polyvinylpyrrolidone;
j) polyalkylenes;
k) aromatic polymers;
l) polyacrylic acids;
m) polyacrylamides;
n) polycyanoacrylates;
o) phenoxyacetic acid/formaldehyde resins;
p) cellulose derivatives;
q) animal, vegetable or synthetic fats and modified fats;
r) animal and vegetable waxes or chemically modified animal and vegetable waxes;
s) animal and vegetable proteins;
t) mono- and disaccharides, oligosaccharides, polysaccharides;
u) vegetable oils, synthetic or semisynthetic oils and animal oils;
v) hardened (hydrogenated or partially hydrogenated) oils/fats;
w) lacquer coatings;
x) fatty acids;
y) silicas;
or mixtures thereof.

10. A formulation as claimed in any of claims 1 to 8, wherein the choline ascorbate is embedded in a matrix which comprises at least one compound as defined in claim 9 which is suitable for forming a matrix which is solid at a temperature in the range from about 20 to 100° C.

11. A formulation as claimed in any of claims 1 to 8, which comprises a porous carrier selected from silicates.

12. A process for preparing a choline ascorbate-containing formulation as claimed in any of the preceding claims, which comprises solid choline ascorbate particles being coated by being
a. sprayed in a fluidized bed with a melt, a solution or a dispersion of a coating composition as defined in claim 9, or subjected to a powder coating with the coating composition in a fluidized bed; or
b. coated in a mixer with a melt, a solution or a dispersion of the coating composition, or subjected to a powder coating with the coating composition; or
c. mixed with fat, and the fat being melted by mechanical energy input and/or heating, while mixing is continued;
and the coated material obtained in each case where appropriate being dried, cooled and/or freed of coarse fractions.

13. A process for preparing a choline ascorbate-containing formulation as claimed in any of claims 1 to 8, which comprises solid choline ascorbate particles being suspended in a melt comprising a (fusible) coating composition as defined in claim 9, and the suspension obtained in this way being dispersed and subsequently solidified.

14. A process for preparing a choline ascorbate-containing formulation as claimed in any of claims 1 to 8, which comprises solid choline ascorbate particles being dispersed in a lipophilic environment, the solid/oil droplets obtained in this way being emulsified in an aqueous phase, and the emulsion being spray-formulated.

15. A process for preparing a choline ascorbate-containing formulation as claimed in any of claims 1 to 8, which comprises choline ascorbate particles being coated by coacervation.

16. A process for preparing a choline ascorbate-containing formulation as claimed in any of claims 1 to 8, which comprises an aqueous protective colloid solution being prepared, choline ascorbate being dissolved or dispersed therein, and the resulting mixture subsequently being spray-formulated or spray-dried and subsequently coated where appropriate.

17. A process for preparing a choline ascorbate-containing formulation as claimed in any of claims 1 to 8, which comprises an aqueous choline ascorbate-containing solution being spray-dried in a fluidized bed and being granulated or agglomerated by addition of suitable additives.

18. A process for preparing a choline ascorbate-containing formulation as claimed in any of claims 1 to 8, which comprises a solution, emulsion or suspension comprising choline ascorbate being mixed with a porous carrier and dried where appropriate; or a melt comprising choline ascorbate being applied to the porous carrier.

19. A process for preparing a choline ascorbate-containing formulation as claimed in any of claims 1 to 8, which comprises wet granules comprising a choline ascorbate-containing solution or dispersion or a choline ascorbate-containing melt and a carrier, or comprising solid, crystalline or amorphous choline ascorbate, being prepared, the wet granules being extruded, where appropriate after-treated, dried and subsequently coated where appropriate.

20. A process for preparing a choline ascorbate-containing formulation as claimed in any of claims 1 to 8, which comprises an aqueous solution of choline ascorbate being prepared, the latter being emulsified in a hydrophobic melt, and the emulsion being solidified.

21. A process for preparing a choline ascorbate-containing formulation as claimed in any of claims 1 to 8, which comprises a melt comprising choline ascorbate being atomized where appropriate in the presence of a dusting agent in a stream of cold gas.

22. A human or animal food which, besides conventional ingredients of human or animal foods, comprises a choline ascorbate-containing formulation as defined in any of claims 1 to 11 in a proportion of about 0.001 to 50% by weight.

23. A human or animal food supplement which, besides conventional ingredients of human or animal food supplements, comprises a choline ascorbate-containing formulation as defined in any of claims 1 to 11 in a proportion of about 0.01 to 99.9% by weight.

24. A pharmaceutical in solid, liquid or pasty form, which comprises in a pharmaceutically suitable carrier an effective amount of a choline ascorbate-containing formulation as claimed in any of claims 1 to 11.

25. The use of a choline ascorbate-containing formulation as claimed in any of claims 1 to 11 for preparing human and animal foods, and human and animal food supplements, or pharmaceuticals.
